(19) United States
(12) Patent Application Publication
(10) Pub. No.: US 2023/0165448 A1
(43) Pub. Date: Jun. 1, 2023

(54) CONTROL OF INGESTIBLE CIRCUITS USING COATINGS

(71) Applicant: INTERNATIONAL BUSINESS MACHINES CORPORATION, Armonk, NY (US)

(72) Inventors: Soma Shekar NAGANNA, Bangalore (IN); Sarbajit K. RAKSHIT, Kolkata (IN); Abhishek SETH, Deoband (IN); Subramanian PALANIAPPAN, TRICHY (IN)

(21) Appl. No.: 17/539,680

(22) Filed: Dec. 1, 2021

(51) Int. Cl. A61B 1/04; A61B 5/06; A61B 5/07; G16H 70/40

(52) U.S. Cl. CPC A61B 1/041; A61B 5/06; A61B 5/073; G16H 70/40

(57) ABSTRACT

A method, including: transmitting, by a first circuit to outside a user's body, information related to a first medication ingested by the user, the first circuit being digestible by gastric juices of the user, the first circuit being covered by a first thickness of a first digestible material; and transmitting, by a second circuit to outside the user's body, information related to a second medication ingested by the user, the second circuit being digestible by the gastric juices of the user, the second circuit being covered by a second thickness of a second digestible material and the first thickness of the first digestible material.

### BACKGROUND

Aspects of the present invention relate generally to ingestible circuits and, more particularly, to controlling the timing of activation of ingestible circuits using a coating on the ingestible circuits.

There are ingestible cameras for gastrointestinal surgeries as well as sensors attached to medications used to study how drugs are broken down in the body. The ingestible sensor may help researchers and doctors more effectively study gastrointestinal diseases. A digestible capsule can be swallowed by a patient and the capsule dissolves in the stomach. Some ingestible circuits include a piezoelectric circuit which generates power when it is exposed to gastric juices. Some ingestible circuits are made using the same material used for making vitamins.

### SUMMARY

In a first aspect of the invention, there is a method including: transmitting, by a first circuit to outside a user's body, information related to a first medication ingested by the user, the first circuit being digestible by gastric juices of the user, the first circuit being covered by a first thickness of a first digestible material; and transmitting, by a second circuit to outside the user's body, information related to a second medication ingested by the user, the second circuit being digestible by the gastric juices of the user, the second circuit being covered by a second thickness of a second digestible material and the first thickness of the first digestible material.

In another aspect of the invention, there is an ingestible pill, the pill including: one or more materials that are digestible by gastric juices of a user; a first circuit, the first circuit being digestible by the gastric juices of the user and being configured to transmit to outside the user's body information related to a first medication ingested by the user; a first thickness of a first digestible material of the materials, the first circuit being covered by the first thickness of the first digestible material of the materials; a second circuit, the second circuit being digestible by the gastric juices of the user and being configured to transmit to outside the user's body information related to a second medication ingested by the user; a second thickness of a second digestible material of the materials, the second circuit being covered by the first thickness of the first digestible material and the second thickness of the second digestible material; and a surface circuit, the surface circuit being digestible by the gastric juices of the user and being configured to transmit to outside the user's body information related to the pill, the surface circuit being located on an outer surface of the first thickness of the first digestible material.

In another aspect of the invention, there is an ingestible pill, the pill including: one or more materials that are digestible by gastric juices of a user; a first circuit, the first circuit being digestible by the gastric juices of the user and being configured to transmit to outside the user's body information related to a first medication ingested by the user; a first thickness of a first material of the materials, the first circuit being covered by the first thickness of the first material of the materials; a second circuit, the second circuit being digestible by the gastric juices of the user and being configured to transmit to outside the user's body information related to a second medication ingested by the user; and a second thickness of a second material of the materials, the second circuit being covered by the first thickness of the first material and the second thickness of the second material.

### BRIEF DESCRIPTION OF THE DRAWINGS

Aspects of the present invention are described in the detailed description which follows, in reference to the noted plurality of drawings by way of non-limiting examples of exemplary embodiments of the present invention.

FIG. 1 depicts a computer infrastructure according to an embodiment of the present invention.

FIG. 2 shows a schematic diagram of an exemplary environment in accordance with aspects of the invention.

FIG. 3 shows a schematic diagram of an exemplary method in accordance with aspects of the invention.

FIG. 4 shows a flowchart of an exemplary method in accordance with aspects of the invention.

### DETAILED DESCRIPTION

Aspects of the present invention relate generally to ingestible circuits and, more particularly, to controlling the timing of activation of ingestible circuits using a coating on the ingestible circuits. According to aspects of the invention, a pill or other ingestible item has one or more ingestible circuits embedded at different depths within the pill or item. The circuits transmit information that is related to one or more medications to a location outside of the user's body. In embodiments, a first ingestible circuit is under a first thickness of a digestible material that is digested by gastric juices (such as, for example, stomach acid) of the user. In this manner, implementations of the invention provide a predetermined timing of the transmission of the information. By using different thicknesses of the digestible material and/or using materials that digest at different rates, embodiments of the invention provide predetermined different timing of transmission of the information from two or more ingestible circuits.

It should be understood that, to the extent implementations of the invention collect, store, or employ personal information provided by or obtained from individuals such information shall be used in accordance with all applicable laws concerning protection of personal information. Additionally, the collection, storage, and use of such information may be subject to consent of the individual to such activity, for example, through “opt-in” or “opt-out” processes as may be appropriate for the situation and type of information. Storage and use of personal information may be in an appropriately secure manner reflective of the type of information, for example, through various encryption and anonymization techniques for particularly sensitive information.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium or media, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

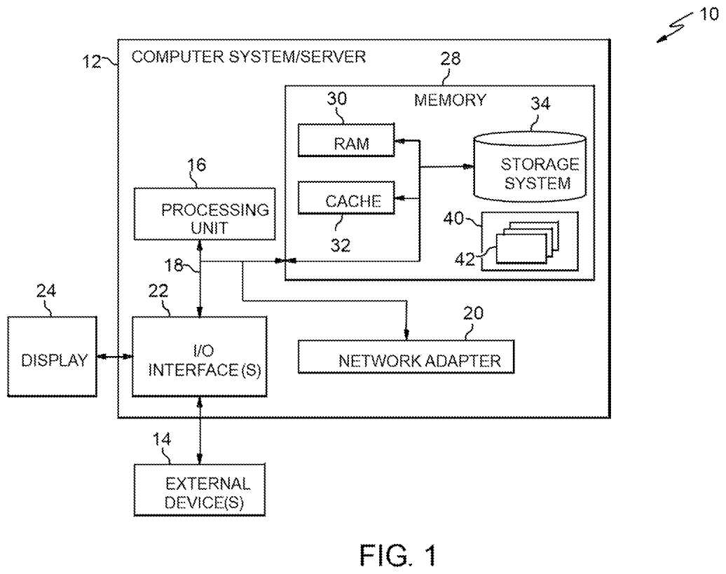

Referring now to FIG. 1, a schematic of an example of a computer infrastructure is shown. Computer infrastructure 10 is only one example of a suitable computer infrastructure and is not intended to suggest any limitation as to the scope of use or functionality of embodiments of the invention described herein. Regardless, computer infrastructure 10 is capable of being implemented and/or performing any of the functionality set forth hereinabove.

In computer infrastructure 10 there is a computer system 12, which is operational with numerous other general purpose or special purpose computing system environments or configurations. Examples of well-known computing systems, environments, and/or configurations that may be suitable for use with computer system 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system 12 may be described in the general context of computer system executable instructions, such as program modules, being executed by a computer system. Generally, program modules may include routines, programs, objects, components, logic, data structures, and so on that perform particular tasks or implement particular abstract data types. Computer system 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system 12 in computer infrastructure 10 is shown in the form of a general-purpose computing device. The components of computer system 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system 12 typically includes a variety of computer system readable media. Such media may be any available media that is accessible by computer system 12, and it includes both volatile and non-volatile media, removable and non-removable media.

System memory 28 can include computer system readable media in the form of volatile memory, such as random access memory (RAM) 30 and/or cache memory 32. Computer system 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system 12 may also communicate with one or more external devices 14 such as a keyboard, a pointing device, a display 24, etc.; one or more devices that enable a user to interact with computer system 12; and/or any devices (e.g., network card, modem, etc.) that enable computer system 12 to communicate with one or more other computing devices. Such communication can occur via Input/Output (I/O) interfaces 22. Still yet, computer system 12 can communicate with one or more networks such as a local area network (LAN), a general wide area network (WAN), and/or a public network (e.g., the Internet) via network adapter 20. As depicted, network adapter 20 communicates with the other components of computer system 12 via bus 18. It should be understood that although not shown, other hardware and/or software components could be used in conjunction with computer system 12. Examples, include, but are not limited to: microcode, device drivers, redundant processing units, external disk drive arrays, RAID systems, tape drives, and data archival storage systems, etc.

Embodiments of the invention include methods and systems by which ingestible circuits (also called edible circuits) are embedded on different layers of any medicine (e.g. capsule) or are mixed with medicine (e.g. syrup) with appropriate types of coating around the ingestible circuit. In embodiments, when a patient (user) swallows such medicine and a portion of the coating dissolves, the ingestible circuit sends a signal to surrounding electronic devices for a duration of side effects of the medicine. In embodiments, one or more automated devices and/or actions are executed in the patient's surroundings.

Different medicines have different side effects, such as, for example, drowsiness, weakness, and/or other side effects. These side effects can adversely affect a user of the medicine. Embodiments provide a benefit to the user such that ingestible circuits embedded with/within the medicine transmit signals/information which identify, for example, the duration of the side effects of the medicine. After taking medicine, body parameters of a patient can change and, because of those changes in the body parameters, in many cases the patient should be restricted from performing specific activities (like driving a vehicle). Also, it is sometimes beneficial to provide an alert to particular persons in the vicinity of the user, to automatically controlling environmental parameters, to restrict privileges of the user, and to provide other notifications or controls.

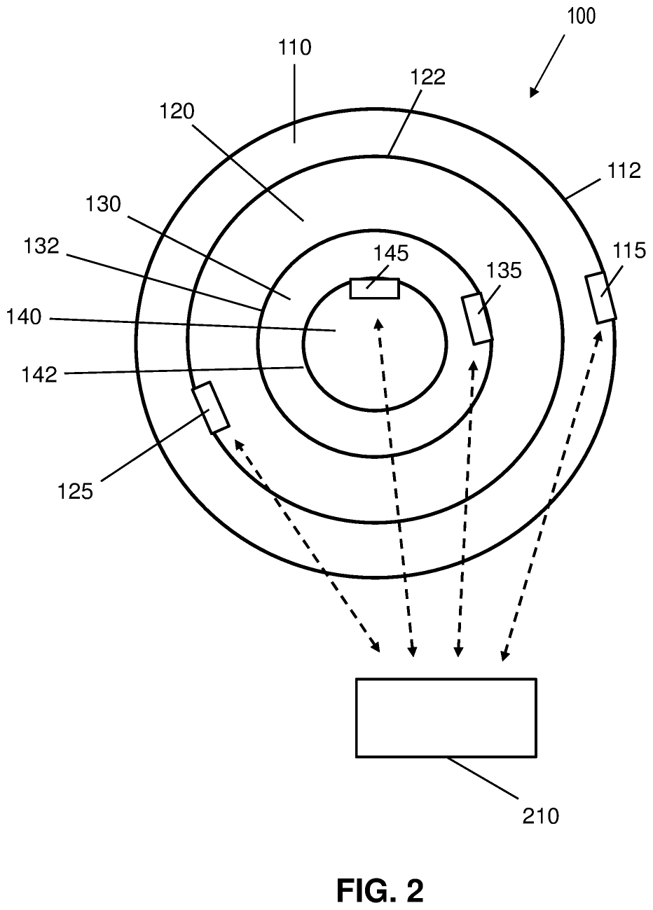

In embodiments, the medicine is designed in such away a way that the embedded ingestible circuit continues to send electronic signals to surrounding electronic devices for the duration which the user is having side effects. Accordingly, for the entire duration of side effects of the patient, the environment is controlled automatically, and appropriate restrictions and notifications will be applied for the user. In embodiments, the signals are transmitted over Bluetooth directly to one or more external devices (such as, for example, external device 210 shown in FIG. 2). Embodiments transmit the signals over other types of wireless communications.

Embodiments provide personalized medicine to a patient by analyzing patient parameters (height, weight, age, vital signs) and medical history of the patient. Embodiments provide personalized medicine having an appropriate number of ingestible electronic circuits mixed with the medicine such that the ingestible circuits send electronic signals to surrounding electronic devices for the entire duration of immediate side effects of the medicine. In this disclosure, the term “immediate side effect” refers to a side effect that is experienced by the patient (user) while the medicine is in the patient's system. Using historical learning, embodiments predict the duration of immediate side effects of any medicine considering the quantity of medicine, type of medicine, patient parameters, and medical history of the patient. Accordingly, personalized medicine is designed in such a way that immediate site effects of the medicine are tracked with the ingestible circuits.

Based on a predicted duration of immediate side effects of any medicine, embodiments provide appropriate layers of coatings applied around the ingestible circuits so that one or more of the ingestible circuits is active for the entire duration of the immediate side effects and continues to send signals to surrounding electronic devices. In embodiments, no individual one of the ingestible circuits is active for the entire duration of the immediate side effects, but over the entire duration of the immediate side effects at least one of the ingestible circuits is active. In embodiments, as a result of the signals from the ingestible circuits over the entire duration of the immediate side effects of medicine, the notifications to the surrounding electronic devices ensure that the patient receives the desired privileges, authentication, priority, restriction, and environment control for the entire duration of immediate side effects of the medicine.

FIG. 2 shows a block diagram of an exemplary environment in accordance with aspects of the invention. In embodiments, the environment includes a pill (or capsule) 100 having a plurality of layers of a digestible material in which a plurality of edible circuits are embedded. The Figures are described with respect to an edible circuit with the terms edible circuit and ingestible circuit being used interchangeably in this disclosure. In the example shown in FIG. 2, pill 100 has a first layer 110 of a first digestible material as the outer most layer of material in pill 100. In this example, a surface edible circuit 115 is embedded in (or attached to) a surface 112 of first layer 110. In this example, pill 100 has a second layer 120 of a second digestible material as the second layer of material in pill 100. In this example, a first edible circuit 125 is embedded in (or attached to) a surface 122 of second layer 120. In this example, pill 100 has a third layer 130 of a third digestible material as the third layer of material in pill 100. In this example, a second edible circuit 135 is embedded in (or attached to) a surface 132 of third layer 130. In this example, pill 100 has a fourth layer 140 of a fourth digestible material as the fourth layer of material in pill 100. In this example, a third edible circuit 145 is embedded in (or attached to) a surface 142 of fourth layer 140. In embodiments, one, some, or all of first layer 110, second layer 120, third layer 130, and fourth layer 140 are the same digestible material. In embodiments, one or more of first layer 110, second layer 120, third layer 130, and fourth layer 140 are one or more medicines.

In embodiments, one or more of edible circuits 115, 125, 135, 145 is a laser-applied graphene circuit including a fuel cell that stores energy to power the circuit. In embodiments, nanobots or materials used in vitamins are used for edible circuits 115, 125, 135, 145. In embodiments, power for the circuit is provided by a galvanic cell that generates a potential in the stomach tract, or by solid electrolyte batteries and supercapacitors built up with biopolymers and edible ions. In embodiments, simple passive radio frequency (RF) systems built up with certificated edible conductors are used to power a simple circuit, either exploiting near field communication (NFC) or RF identification (RFID) protocols for the data transmission. In embodiments, acoustic communication based on piezoelectric transducers is utilized. Embodiments include active layers that are thin films of magnesium and cuprous chloride. In these embodiments, a gold underlayer acts as a current collector underneath the cuprous chloride, and upon contact with gastric fluid, these layers create a battery that activates and powers the circuit.

In one exemplary embodiment, pill 100 does not include surface edible circuit 115 and, as a result, first layer 110 covers first edible circuit 125 as the outermost edible circuit. In such embodiments, first layer 110 provides a time delay before first edible circuit 125 begins transmitting. Also shown in FIG. 2 is an external device 210 which is located outside of a user's body and is configured to receive signals from one or more of edible circuits 115, 125, 135, 145. In embodiments, external device 210 is a computing device and comprise one or more elements of computer system 12 of FIG. 1. Examples of external device 210 include a smart watch, a smart phone, a notification device (such as a warning light) in a vehicle, a residential or vehicular thermostat, and a monitoring device in a hospital or other facility.

In embodiments, in response to edible circuit 115, 125, 135, 145 being exposed to gastric juices (stomach acid) of the user after being ingested by the user, the edible circuit generates power and communicates with external device 210. In embodiments, edible circuits 115, 125, 135, 145 start dissolving when contacted by gastric juices and gradually dissolve while they are exposed to gastric juices, or in contact with another body fluid. In embodiments, side effects of different medicines are analyzed to identify the duration of side effects for a person of given parameters. Embodiments consider, during the analysis, patient specific parameters such as, for example, age, weight, and current health condition, as well as the historical medical profile of the patient. Embodiments consider, during the analysis, external influencing factors which effect the side effect such as, for example, weather parameters and surrounding condition. Embodiments include historical learning to understand the duration of side effects of the patient that result from a given medicine and to identifying the degree (extent) of side effects over the duration of the side effects. Embodiments consider the duration of side effects based on a quantity of the medicine. Embodiments consider the corpus of the above-listed knowledge (and other knowledge) to predict the duration and degree of immediate side effects of the patient after taking the medication and determining the material to be used and the thickness of each level 110, 120, 130, 140.

In embodiments, in the manner described above, edible circuits 115, 125, 135, 145 generate electric signals once they are exposed to gastric juices. In embodiments, when a particular layer of material (coating) is dissolved, an edible circuit will be exposed to gastric juices and, after a certain duration, the edible circuit will be dissolved and will stop sending signals.

In embodiments, external device 210 is an Internet of Things (IoT) device and analyzes the signal from edible circuits 115, 125, 135, 145 along with, and in relation to, information from other sources such as, for example, weather information, location information, and other information that might be relevant to side effect duration and/or safety of the user. In embodiments, external device 210 controls environmental automation such as, for example, temperature control. In embodiments, external device 210 includes an alarm/notification to notify the user that it is time to take a dose of the same or a different medicine.

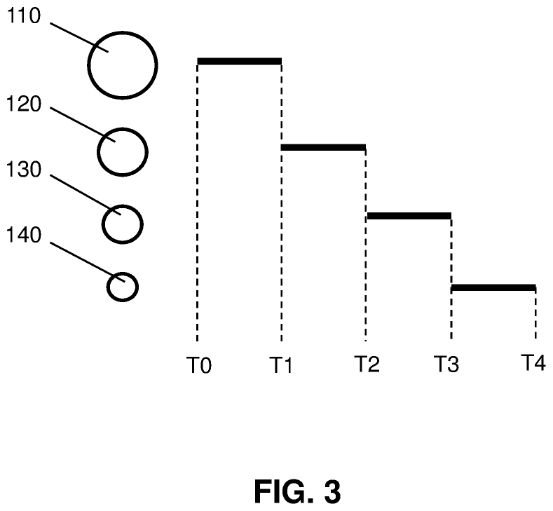

Referring to the exemplary embodiment shown in FIGS. 2 and 3, a user ingests pill 100 at time T0. In embodiments that include surface edible circuit 115, edible circuit 115 is energized by contacting the gastric juices of the user and begins sending a signal to external device 210. As time progresses from time T0 to time T1, first layer 110 dissolves and surface edible circuit 115 transmits. At time T1, first layer 110 is sufficiently dissolved to expose second layer 120 and first edible circuit 125 to the gastric juices. As time progresses from time T1 to time T2, second layer 120 dissolves and first edible circuit 125 transmits. At time T2, second layer 120 is sufficiently dissolved to expose third layer 130 and second edible circuit 135 to the gastric juices. As time progresses from time T2 to time T3, third layer 130 dissolves and second edible circuit 135 transmits. At time T3, third layer 130 is sufficiently dissolved to expose fourth layer 140 and third edible circuit 145 to the gastric juices. As time progresses from time T3 to time T4, fourth layer 140 dissolves and third edible circuit 145 transmits.

In embodiments, surface edible circuit 115 transmits for the same duration as first layer 110 takes to dissolve, first edible circuit 125 transmits for the same duration as second layer 120 takes to dissolve, second edible circuit 135 transmits for the same duration as third layer 130 takes to dissolve, and third edible circuit 145 transmits for the same duration as fourth layer 140 takes to dissolve. In other embodiments, one or more of edible circuits 115, 125, 135, 145 transmits for a duration longer than the period of time its corresponding layer takes to dissolve, resulting in an overlap of one or more of the transmission periods.

In embodiments, edible circuits 115, 125, 135, 145 transmit the same type of information, such as, for example, that the medicine is being dispensed (the layer is being dissolved). In other embodiments, one of more of edible circuits 115, 125, 135, 145 transmit different types of information. For example, in embodiments, edible circuits 115, 125 and 135 transmit information that alerts external device 210 that the medicine is being dispensed, and edible circuit 145 transmits information that instructs external device 210 to decrease the temperature in the environment of the user. Embodiments provide many different combinations of transmitted instructions and timing of those instructions.

In embodiments, layers 110, 120, 130, 140 are the medication being administered to the user. In other embodiments, one or more of layers 110, 120, 130, 140 are layers of inert material that, through their dissolving, establish timing of the activation and duration of edible circuits 115, 125, 135, 145. For example, in embodiments, pill 100 contains no medicine and is solely a timing device to transmit timing information regarding a separately taken medication.

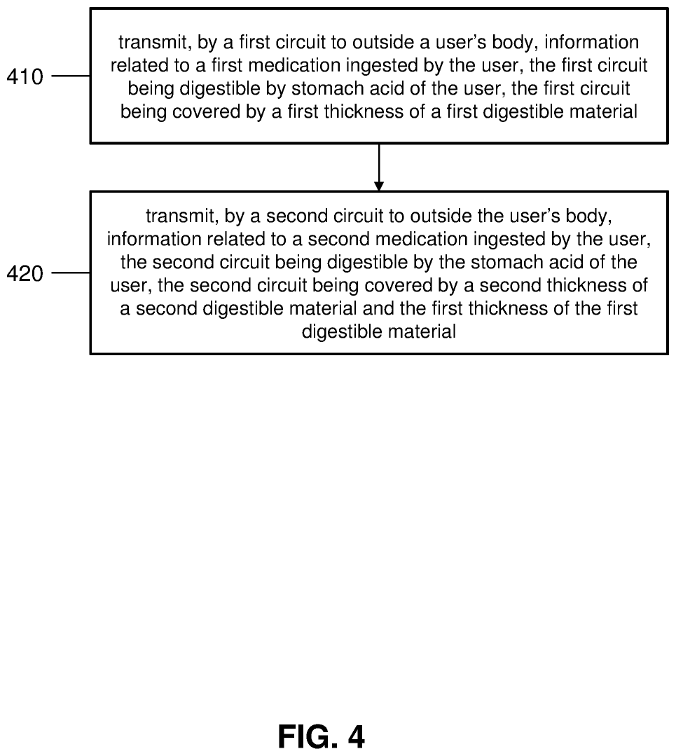

FIG. 4 shows a flowchart of an exemplary method in accordance with aspects of the present invention. Steps of the method may be carried out in the environment of FIG. 2 and are described with reference to elements depicted in FIG. 2.

At step 410, the system transmits, by a first circuit to outside a user's body, information related to a first medication ingested by the user, the first circuit being digestible by stomach acid of the user, the first circuit being covered by a first thickness of a first digestible material. In embodiments, and as described with respect to FIGS. 2 and 3, pill 100 transmits, by first circuit 125 to outside a user's body, information related to a first medication ingested by the user, first circuit 125 being digestible by stomach acid of the user, first circuit 125 being covered by a first thickness of first layer 110. In embodiments, external device 210 is configured to receive the information transmitted by first circuit 125 and then performs an operation based on the information.

At step 420, the system transmits, by a second circuit to outside a user's body, information related to a second medication ingested by the user, the second circuit being digestible by stomach acid of the user, the second circuit being covered by a second thickness of a second digestible material and the first thickness of the first digestible material. In embodiments, and as described with respect to FIGS. 2 and 3, pill 100 transmits, by second circuit 135 to outside a user's body, information related to a second medication ingested by the user, second circuit 135 being digestible by stomach acid of the user, second circuit 135 being covered by a second thickness of second layer 120 and the first thickness of first layer 110. In embodiments, external device 210 is configured to receive the information transmitted by second circuit 135 and then performs an operation based on the information. In embodiments the operation performed based on the information transmitted by second circuit 135 is the same operation or a different operation than the operation performed based on the information transmitted by first circuit 125.

In embodiments, a service provider could offer to perform the processes described herein. In this case, the service provider can create, maintain, deploy, support, etc., the computer infrastructure that performs the process steps of the invention for one or more customers. These customers may be, for example, any business that uses technology. In return, the service provider can receive payment from the customer(s) under a subscription and/or fee agreement and/or the service provider can receive payment from the sale of advertising content to one or more third parties.

In still additional embodiments, the invention provides a computer-implemented method, via a network. In this case, a computer infrastructure, such as computer system 12 (FIG. 1), can be provided and one or more systems for performing the processes of the invention can be obtained (e.g., created, purchased, used, modified, etc.) and deployed to the computer infrastructure. To this extent, the deployment of a system can comprise one or more of: (1) installing program code on a computing device, such as computer system 12 (as shown in FIG. 1), from a computer-readable medium; (2) adding one or more computing devices to the computer infrastructure; and (3) incorporating and/or modifying one or more existing systems of the computer infrastructure to enable the computer infrastructure to perform the processes of the invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
transmitting, by a first circuit to outside a user's body, information related to a first medication ingested by the user, the first circuit being digestible by gastric juices of the user, the first circuit being covered by a first thickness of a first digestible material; and
transmitting, by a second circuit to outside the user's body, information related to a second medication ingested by the user, the second circuit being digestible by the gastric juices of the user, the second circuit being covered by a second thickness of a second digestible material and the first thickness of the first digestible material.

2. The method of claim 1, wherein the first digestible material and the second digestible material are the same material.

3. The method of claim 1, wherein the information related to a first medication comprises a notification that the user has ingested the first medication.

4. The method of claim 3, wherein the first digestible material is the first medication.

5. The method of claim 4, wherein the second digestible material is the first medication.

6. The method of claim 3, wherein the second digestible material is a second medication, the second medication and the first medication being different medications.

7. The method of claim 3, wherein the first digestible material is the first medication.

8. The method of claim 1, wherein the transmitting by the first circuit begins as a result of the first circuit being contacted by the gastric juices of the user.

9. The method of claim 8, wherein the transmitting by the first circuit ends before the transmitting by the second circuit begins.

10. The method of claim 8 wherein the transmitting by the second circuit begins as a result of the second circuit being contacted by the gastric juices of the user.

11. The method of claim 1, wherein the first thickness and the second thickness are different thicknesses.

12. The method of claim 1, further comprising:
transmitting, by a third circuit to outside the user's body, information related to a third medication ingested by the user, the third circuit being digestible by the gastric juices of the user, the third circuit being covered by a third thickness of a third digestible material, the second thickness of the second digestible material, and the first thickness of the first digestible material.

13. The method of claim 12, wherein the first digestible material, the second digestible material, and the third digestible material are the same material.

14. The method of claim 12, wherein the first digestible material is the first medication, the second digestible material is the second medication, and the third digestible material is the third medication, and
the first medication, the second medication, and the third medication are the same medication.

15. An ingestible pill, the pill comprising:
one or more materials that are digestible by gastric juices of a user;
a first circuit, the first circuit being digestible by the gastric juices of the user and being configured to transmit to outside the user's body information related to a first medication ingested by the user;
a first thickness of a first digestible material of the materials, the first circuit being covered by the first thickness of the first digestible material of the materials;
a second circuit, the second circuit being digestible by the gastric juices of the user and being configured to transmit to outside the user's body information related to a second medication ingested by the user;
a second thickness of a second digestible material of the materials, the second circuit being covered by the first thickness of the first digestible material and the second thickness of the second digestible material; and
a surface circuit, the surface circuit being digestible by the gastric juices of the user and being configured to transmit to outside the user's body information related to the pill, the surface circuit being located on an outer surface of the first thickness of the first digestible material.

16. An ingestible pill, the pill comprising:
one or more materials that are digestible by gastric juices of a user;
a first circuit, the first circuit being digestible by the gastric juices of the user and being configured to transmit to outside the user's body information related to a first medication ingested by the user;
a first thickness of a first digestible material of the materials, the first circuit being covered by the first thickness of the first digestible material of the materials;
a second circuit, the second circuit being digestible by the gastric juices of the user and being configured to transmit to outside the user's body information related to a second medication ingested by the user; and
a second thickness of a second digestible material of the materials, the second circuit being covered by the first thickness of the first digestible material and the second thickness of the second digestible material.

17. The ingestible pill of claim 16, wherein the information related to a first medication comprises a notification that the user has ingested the first medication.

18. The ingestible pill of claim 16, wherein the first digestible material is the first medication.

19. The ingestible pill of claim 16, wherein the first circuit is configured to transmit the information related to the first medication as a result of the first circuit being contacted by the gastric juices of the user.

20. The ingestible pill of claim 16, wherein the second circuit is configured to transmit the information related to the second medication as a result of the second circuit being contacted by the gastric juices of the user.
